SEQUENCE LISTING (9 sequences)

SEQ ID NO: 1 - DNA, 1561 bp, Sus scrofa
  CDS (90)..(1310)
actgtacacatgataggatccagggcagctggaccaaggcagcagttacagccggaacccactgcagaccggcctggccatcccggacaatgctgctcaccctgtatgcctgtctcctctggctgtcgaccagcgggctctggaccagccaggctaaggaccctgacagtgacttgagcacaaggagccgtcaccggaacttggctccaaacaacgtggactttgcctttgccctgtataagcacctggtggcctcagctcctggcaaggacgtcttcctctcccctgtgagcatctccacagccttggctatgctgtcactaggtgccagtggctacacacgggagcagcttctccaaggcctaggcttcaacctcactgagacccccgaagctgagatccatcaggacttccagcatctccactctctcctcaaggggtcaaacatcacctcagaaatgaccatgggcaatgccttgttcctcgaccgcagtctggagcttctggagtccttctccacaggctccaagcactactacgggttggaagctttggctgccgatttccaggactgggcaggagccagcagacaaattaatgagtatatcaagaataagacgcaagggaaaattgtggacttgttcttggagcaggatagctcagccatgctcatcctgatcaactatatcttctttaaaggcacgtggacacactccttccccccagaaagcaccagggaagagaacttctatgtgaacgagacggccacggtcaaggtgcccatgatgttccagtcgcgcgccatgaagtacttgaatgactccttgctcccctgccagctggtgcagctggaatacacgggcaatgagacggccttcttcatcctcccggtcaagggggagatggacacggtcattgccgggctgagccgggacaccattcagaggtggtcgaagtccctgatccccagccaggtggacctgtacgtcccaaaggtctccatctccggagcctatgacctcgggagcatcctgggggacatgggcattgtggacttgctcagccacccaacacacttctcaggcatcacccagaatgccctgccgaagatgtccaaggtggtccacaaggcggttctgcaatttgacgagaagggcatggaggcagctgcccccactacgcgtggacgcagcctgcacgcggcgcccaagcctgtcactgtccacttcaaccggcccttcatcgtcatggttttcgaccacttcacgtggagcagccttttcctgggcaagattgtgaatctgacctaagaggaggcgccttaggaaccacagctcatctgactctggcatcagggaccccaaagaaatgttctgggtggtgggtcgtttcccccagtctctcccaggttctcctgctggaataaatgtcattgcgactgatgccaagtgtggtggaaggggaaggtgggacaactgataaaattagaataatatcaatataatctcaatataatactgtaccaatgtaatttactgggttcgatcgttgtcctagggtt

SEQ ID NO: 2 - protein, 406 aa, Sus scrofa
MLLTLYACLLWLSTSGLWTSQAKDPDSDLSTRSRHRNLAPNNVDFAFALYKHLVASAPGKDVFLSPVSISTALAMLSLGASGYTREQLLQGLGFNLTETPEAEIHQDFQHLHSLLKGSNITSEMTMGNALFLDRSLELLESFSTGSKHYYGLEALAADFQDWAGASRQINEYIKNKTQGKIVDLFLEQDSSAMLILINYIFFKGTWTHSFPPESTREENFYVNETATVKVPMMFQSRAMKYLNDSLLPCQLVQLEYTGNETAFFILPVKGEMDTVIAGLSRDTIQRWSKSLIPSQVDLYVPKVSISGAYDLGSILGDMGIVDLLSHPTHFSGITQNALPKMSKVVHKAVLQFDEKGMEAAAPTTRGRSLHAAPKPVTVHFNRPFIVMVFDHFTWSSLFLGKIVNLT

SEQ ID NO: 3 - DNA, 1090 bp, Sus scrofa
  misc_feature (1)..(1090): Region 5′ flanking the Cbg gene
gccctattcttcagcatactgtatcagtccaaggatgggcaaccatatcctttgagggactgatatgaacactgaaaaaaagtgagtctctctccttctgatatcaaataccaagttagaaccaaataacctggagctgcagtggccacctttgtccccaccacagagagaccaaagaaggaaaccataagaaagcagagttaagaaagaatttacaggggggaaaaaaaaaaaaagagcaagagacagagagacagagatttactgacaagattttgtcccctggatccaaccaggcctgaagcattctccactcttcagaactctccatcagggaaacccaataaattttttttttaaagtagtttgagtttggtctcttgtcacctgcaatcaaaagagaactgatgaatgaaaatgtgaggaagagagaagttgctagacgttgcacagcaacaccagattcttaacccactgagcaagggcagggatcaaacctgccacctcatggttcctagttggattcattaaccactgaactacgacgggaactctgaggttgctacactttaaatggccttagatatgaatctcaagcttaagtttcatgtgtctcagtttcgtttaaagaagagtaataataataatagtatccatttagcgtcatcgtggagattaaattataccatgactgtctttcccactagaccattgctccctccaaggcaggactttgctgaactcattcccagcccaagctcagcgcctggttcttcacaagttatcagcaccatcatgctaattcagggcacctgtgacttctgtgccaggtgccatggggcaaccacaaacattgacgcaactccagccacattttgatattaaaaactaaaatgtcagggaattgtatacagaagaaagttaaatcacagtattgctgtgtttactcaaatttcctgggaaagcttcccattggttaatgactggggggaggggaccaccaaatggtgaaggccactggcccccctctaaccattaaccagcagggaagctggcaaacaagatttagcggcagcggccccgcagactt

SEQ ID NO: 4 - DNA, 20 bp, Artificial Sequence
  Direct primer, stemming from exon 2 of the human Cbg gene
acacctgtcttctctggctg

SEQ ID NO: 5 - DNA, 19 bp, Artificial Sequence
  Reverse primer, stemming from exon 2 of the human Cbg gene
acaggctgaaggcaaagtc

SEQ ID NO: 6 - DNA, 86 bp, Sus scrofa
  intron (1)..(86): Intron C of the porcine Cbg gene. Part 5′
aggtaagctctccagcagcccgtggtgatttctgcagtggatgcgtacctggaaagcagggcgagtaggaaaggggtgcacctggc

SEQ ID NO: 7 - DNA, 133 bp, Sus scrofa
  intron (1)..(133): Intron C of the porcine Cbg gene. Part 3′
cctcactaaaatatctaaccagcattaacctatagcatgtgccatattctggtctggagggagcacaggggaaacgggagtctggctgccaggcccagccctgggagatctaaccccatctgctnactccgag

SEQ ID NO: 8 - DNA, 20 bp, Artificial Sequence
  Sense primer
ccctgtatgcctgtctcctc

SEQ ID NO: 9 - DNA, 20 bp, Artificial Sequence
  Antisense primer
cctgctccaagaacaagtcc

(19) United States
(12) Patent Application Publication
(10) Pub. No.: US 2008/0115236 A1
(43) Pub. Date: May 15, 2008

(54) CBG gene as a genetic marker of hypercortisolism and associated pathologies

(75) Inventors: Marie-Pierre Moisan, Bordeaux (FR); Pierre Mormede, Merignac (FR); Denis Milan, LaBege (FR); Jean-Pierre Bidanel, Palaiseau (FR); Olga Ousova, Bordeaux (FR)

Correspondence Address: IP GROUP OF DLA PIPER US LLP, ONE LIBERTY PLACE, 1650 MARKET ST, SUITE 4900, PHILADELPHIA, PA, 19103, US

(73) Assignee: Institut National de la Recherche Agronomique (INRA), a corporation of France, Paris Cedex (FR)

(21) Appl. No.: 11/890,368

(22) Filed: Aug. 6, 2007

Related U.S. Application Data
(62) Division of application No. 10/833,970, filed on Apr. 28, 2004, now abandoned.
Application No. 10/833,970 is a continuation of application No. PCT/FR02/03762, filed on Oct. 31, 2002.

(30) Foreign Application Priority Data
Oct. 31, 2001 (FR) FR 01/14156
Jul. 26, 2002 (FR) FR 02/09551

(51) Int. Cl. A01K 67/027; C12Q 1/68; C12P 19/34

(52) U.S. Cl. 800/14; 435/6; 435/91.2

(57) ABSTRACT

A method for identifying polymorphic markers associated with a hypercortisolism phenotype including comparing nucleic acid sequences, from multiple individuals, including all or part of a Cbg gene; and identifying mutations in the Cbg gene or sequences adjacent to it.

### RELATED APPLICATION

This is a divisional of application Ser. No. 10/833,970, filed Apr. 28, 2004, which is a continuation of international Application No. PCT/FR02/03762, with an international filing date of Oct. 31, 2002 (WO 03/038124, published May 8, 2003), which is based on French Patent Application Nos. 01/14156, filed Oct. 31, 2001, and 02/09551, filed Jul. 26, 2002, incorporated herein by reference.

### FIELD OF THE INVENTION

This invention pertains to the gene coding for transcortin (or CBG=corticosteroid-binding globulin) as a genetic marker of constitutive hypercortisolism and as a new therapeutic target of pathologies associated with hypercortisolism. The invention has as applications: 1) selection of breeding animals having a lower probability of developing hypercortisolism; 2) genetic diagnosis of patients susceptible to developing, hypercortisolism; and 3) treatment of pathologies linked to constitutive hypercortisolism. The invention also relates to methods for identifying the genetic markers of transcortin and genetic screening methods for determining individuals susceptible to developing hypercortisolism.

### BACKGROUND

The glucocorticoid hormones, cortisol in man and pigs, corticosterone in rodents, are implicated in numerous biological processes such as neoglucogenesis, lipid and protein metabolism, anti-inflammatory action and growth. The glucocorticoids are also a major component of stress responses. After exposure to a stress, cortisol is rapidly liberated from the suprarenal glands to provide the energy required for the behavioral response. By negative retrocontrol, the cortisol level returns to the baseline values when this stimulus has been controlled by the individual. In the contrary case, such as chronic stress situations, the constant, elevated levels of cortisol have an intensely deleterious impact on the organism. Thus, cortisol and the corticotropic axis in general are implicated in diverse pathologies such as obesity (Rosmond et al., 1998), constitutive sensitivity to inflammatory and autoimmune reactions (Sternberg and Gold, 1997), aging (Lupien et al., 1998) and sensitization to drugs of abuse (Piazza and Le Moal, 1998).

A noteworthy variability in the functioning of the corticotropic axis is seen between individuals, which influences the individual vulnerability to the pathologies cited above. This variability is in part of genetic origin as attested to be multiple twin studies focused on the reactivity of the corticotropic axis to stress and its circadian activity (Meikle et al., 1998; Kirschbaum et al., 1992; Linkowski et al., 1993). Similarly, the functional differences of the corticotropic axis have been demonstrated between diverse consanguineous lines of mice and rats (Armario et al., 1995; Marissal-Arvy et al., 1999) and between breeds of pigs (Désautés et al., 1999).

Identification of the genes supporting this variability in the functioning of the corticotropic axis is, therefore, of great importance for human and animal health. In humans, studies have been performed on the association between the regulator genes of the corticotropic axis and the pathologies linked to the dysfunction of this axis. For example, polymorphisms of the glucocorticoid receptor have been associated with abdominal obesity (Buemann et al., 1997).

### SUMMARY OF THE INVENTION

This invention relates to a method for identifying polymorphic markers associated with a hypercortisolism phenotype including comparing nucleic acid sequences, from multiple individuals, including all or part of a Cbg gene; and identifying mutations in the Cbg gene or sequences adjacent to it.

This invention also relates to a polymorphic marker responsible for a hypercortisolism phenotype including all or part of a nucleic acid sequence including a Cbg gene or adjacent 3′ or 5′ sequences distanced apart by no more than about 100 kb.

This, invention further relates to a nucleotide primer including from about 5 to about 50 successive nucleotides of a sequence of the Cbg gene or the adjacent 3′ or 5′ sequences distanced apart by no more than about 100 kb, flanking a polymorphic marker.

This invention still further relates to a genetic screening method for identifying individuals susceptible to developing hypercortisolism and associated pathologies with polymorphic markers including i) purifying genomic DNA from an individual, ii) amplifying a locus containing a polymorphic marker by PCR from the DNA, and iii) detecting allele(s) of the polymorphic marker in the amplified DNA.

This invention yet further relates to a kit for testing genetic markers of hypercortisolism from a DNA sample including a pair of nucleotide primers; PCR reagents; and one of negative and positive controls of reactions and markers.

This invention also further relates to a method of diagnosing a hypercortisolism or a predisposition to a hypercortisolism in a subject enabling identification of a dysfunction of the corticotropic axis and a disease or a predisposition to a disease linked to this axis including i) purifying genomic DNA from an individual, ii) amplifying a locus containing a polymorphic marker by PCR from the DNA, iii) detecting allele(s) of the polymorphic marker in the amplified DNA and iv) determining hypercortisolism or a predisposition, to hypercortisolism based on the presence or absence of said allele(s).

This invention still yet further relates to a transgenic animal transgene containing a nucleic sequence, overexpressing a sequence according to a nucleic sequence and coding for a polypeptide identical to or homologous with the protein CBG, and a method for identifying a compound that modulates a function of CBG protein and reduces a hypercortisolism of a subject including binding the compound to the CBG protein; determining cortisol displacement capacity between the CBG protein and the compound; and selecting compounds exhibiting efficacy relative to cortisol.

### BRIEF DESCRIPTION OF THE DRAWINGS

The file contains at least one drawing executed in color. Copies of this publication with color drawings will be provided by the Office upon request and payment of necessary fee.

Other advantages and characteristics of the invention will become apparent from the examples below which pertain to the identification of mutations in the Cbg gene of the pig and the analysis of genetic links demonstrating a cause and effect relationship between the molecular variants of CBG and the production of cortisol. Reference will be made to the attached figures in which:

FIG. 1 represents the localization of the porcine Cbg gene by mapping on irradiated hybrids. In A: Evolution of the maximal likelihood level along Sscr 7 (in cM) for the concentrations of plasma cortisol. In B: Cartography profile by irradiated hybrids of chromosome 7 of the pig. The distances are in cR₇₀₀₀;

FIG. 2 represents localization of the porcine Cbg gene at 7q26 by FISH;

FIG. 3 represents the analysis of genetic links of the plasma CBG concentrations on 81 F2 pigs;

FIG. 4 represents the detection of mutation in the genomic sequence of Cbg. The arrows indicate the nucleotide for which the F1 pig #9110045 and its Meishan mother are heterozygotes (T/G) whereas the LW father is homozygous (G/G); and

FIG. 5 is a graph showing position (M) versus L value.

### DETAILED DESCRIPTION

We believed that it would be advantageous to identify the genes, relating to the functioning of the corticotropic axis using (i) approaches not posing base hypotheses and using quantitative trait loci (QTL) genetic mapping on animal models (Moisan et al., 1996) and (ii) candidate gene approaches based on the knowledge of the role of these genes in the functioning of the corticotropic axis (Marissal-Arvy et al., 2000).

These two strategies were implemented on the gene coding for transcortin, and the studies leading to this invention involved both a QTL analysis and the known function of this gene. Transcortin or CBG (corticosteroid-binding globulin) is a plasma binding protein of the glucocorticoid hormones which was studied for its role as cortisol transporter and its influence on the bioavailability of cortisol. We were able to demonstrate the role of transcortin on the production of cortisol and its implication in the variability of blood cortisol levels in pigs.

These results were obtained from our studies on the genes influencing the functioning of the corticotropic axis in pigs, and employing the QTL genetic mapping method on the growth of two breeds of pigs: European Large White and Chinese Meishan.

We found a strong genetic link between the plasma concentrations of cortisol in the pigs and a locus of porcine chromosome 7 (Bidanel et al., 2000). By comparative mapping with the human genome, we determined that the gene of transcortin can be found in the interval defined by genetic analysis:

the porcine Cbg gene can be found in the region of the QTL (demonstrated by FISH and by cartography on irradiated hybrids),

genetic link analysis performed with the plasma concentration of transcortin (rather than cortisol) on the same F2 population also shows a strong link with the locus of chromosome 7,

the concentration of CBG is different in the two breeds of parent pigs Large White and Meishan, and

an interesting mutation was found in the coding region of the Cbg gene in the Meishan pig.

The Cbg gene thus constitutes a position candidate for this QTL in pigs. These results of the analysis of genetic links demonstrate, for the first time, a cause and effect relationship between the molecular variants of transcortin and the production of cortisol. Moreover, the Meishan pig, which is characterized by hypercortisolism, is obese and exhibits growth retardation in relation to the Large White which could be a consequence of its high levels of cortisol. In favor of this hypothesis, a positive correlation was found in the F2 individuals between the cortisol level and the thickness of the backfat. This relationship was also found in a Duroc×Large White F2 population between urinary cortisol, backfat and lean meat proportion in the carcass. Following these results, a new statistical analysis demonstrated a genetic link between the thickness of the backfat and the locus of chromosome 7 in the population of F2 pigs (Meishan×Large White).

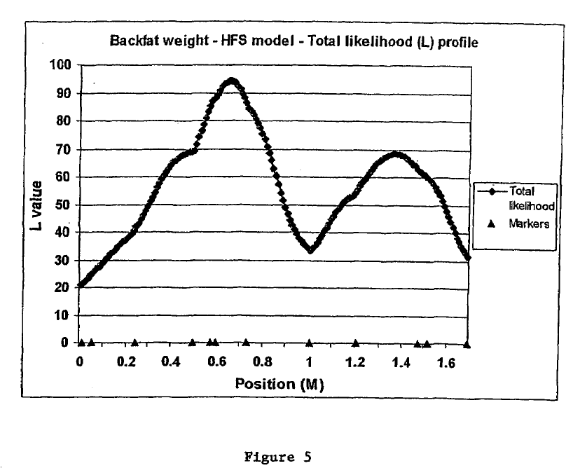

FIG. 5 illustrates the genetic link between the backfat thickness and the QTL of hypercortisolism. The Cbg gene is in Morgan position 1.35 of chromosome 7, at the peak of the second QTL presented. Blood cortisol levels and fat deposition thus converge at this locus containing the transcortin gene. The Meishan pig therefore represents an excellent model for studying the genetic variability of the corticotropic axis and its physiopathological consequences for obesity, in particular.

It is appropriate to recall that in the mouse a genomic locus associated with obesity was demonstrated in 1995 by Warden (Warden et al., 1995). With the present data on the mouse genome, it is possible to see that CBG is at the peak of the confidence interval of this QTL and again constitutes in this model a good position candidate.

Finally, in humans an inverse relationship between the concentration of CBG and hyperinsulinemia was reported in the context of obesity, whereas diabetics have a higher CBG (Fernandez-Real et al., 2000). These relationships could be explained by the inhibitory effect of insulin on the hepatic production of CBG (Crave et al., 1995). Molecular variants of transcortin in humans have been described in the literature (Van Baelen et al., 1982), (Smith et al., 1992) and in two studies the patients having a mutation in the transcortin gene were obese (Emptoz-Bonneton et al., 2000; Torpy et al., 2001).

We used different approaches to consider the variations of CBG in obesity as an important factor in the bioavailability of cortisol implicated in the physiopathology of the disease. These results are remarkable because no marker of constitutive blood cortisol levels is available at present. Such a marker would allow us to determine from a blood sample, and from birth, the blood cortisol level of an individual. This blood cortisol level influences the vulnerability to obesity, autoimmune and inflammatory reactions, growth rate, aging and drug addiction. The invention thus has applications in the field of the selection of breeding animals, such as pigs, carrying favorable alleles of the transcortin gene, as well as for genetic diagnosis in humans of predisposition to constitutive hypercortisolism and its previously mentioned consequences. Finally, the invention makes available a new tool for screening for substances useful for treating these pathologies linked to dysfunction of the corticotropic axis.

Thus, the invention has as object a method for identifying polymorphic markers associated with the hypercortisolism phenotype comprising the comparison of nucleic acid sequences, from multiple individuals, comprising all or part of the Cbg gene and the identification of mutations in the Cbg gene or the sequences adjacent to it.

The term “individuals” is understood to mean human as well as animal subjects. The nucleic acid sequences are advantageously genomic DNA sequences comprising a part of the Cbg gene or an adjacent 3′ or 5′ sequence preferably distanced apart by no more than 100 kb.

As a non-limiting example, the cDNA sequence of the porcine Cbg gene and an adjacent 5′ sequence of this gene, which are represented in the attached sequence listing as numbers SEQ ID NO. 1 and SEQ ID. NO. 3, respectively, can be used to search for hypercortisolism markers.

These markers can be obtained from genomic clones comprising a part of the Cbg gene or flanking sequences, themselves, obtained from a DNA data bank screening with a specific probe of the Cbg gene as described hereinafter. These polymorphic markers can be, for example, microsatellites, insertion/deletion polymorphisms, restriction fragment length polymorphisms (RFLP) or single nucleotide polymorphisms (SNP).

Sequencing of a DNA segment covering the polymorphic locus enables definition of the nucleotide primers enabling the specific amplification of said segment from the total genomic DNA of an individual. Thus, the invention relates to a polymorphic marker associated with the hypercortisolism phenotype constituted of all or part of a nucleic acid sequence comprising the Cbg gene or the adjacent 3′ or 5′ sequences preferably distanced apart by no more than about 100 kb.

The invention also pertains to nucleotide primers flanking the above marker. Such primers comprise from about 5 to about 50, preferably from about 10 to about 30, successive nucleotides of the sequence of the Cbg gene or the adjacent 3′ or 5′ sequences preferably distanced apart by no more than about 100 kb, flanking a marker as defined above.

The invention also pertains to a genetic screening method for identifying individuals susceptible to developing hypercortisolism and associated pathologies by means of polymorphic markers. Such a method comprises:

i) purifying genomic DNA from an individual's blood, tissue or sperm (generally the DNA is purified from the leukocytes from a blood sample obtained by conventional techniques), then

ii) amplifying the locus containing the polymorphic marker by polymerase chain reaction (or PCR) from the DNA by means of the primers defined above, and iii) detecting the allele(s) of the polymorphic marker in the amplified DNA.

Different techniques can be employed depending on the type of polymorphism of the marker:

For the length polymorphisms (microsatellites, insertion/deletion/RFLP), the alleles present in the amplified DNA of different individuals are detected by the conventional techniques of electrophoresis, preferably preceded by an enzymatic digestion for the RFLP.

For the punctiform mutations, SNP-type polymorphisms, the techniques employed include, e.g., SSCP (Single Strand Conformation Polymorphism) (Orita et al., 1989, PNAS 86: 2766-2770), allele-specific PCR (Gibbs 1987, Nucl Acid Res 17, 2427-2448) or direct sequencing of the amplified DNA.

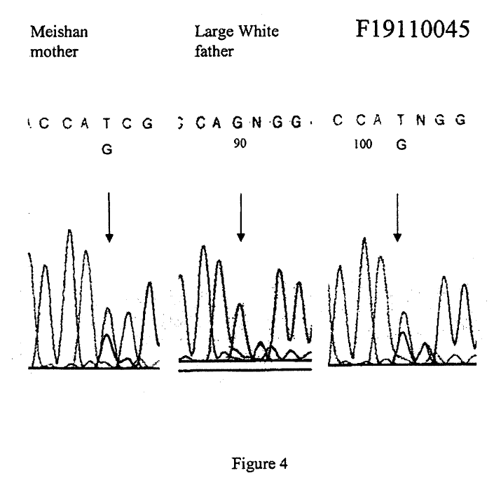

Detection of the alleles of the marker in an individual makes it possible to predict whether the patient is more susceptible to develop hypercortisolism. An example of such a punctiform mutation in the Cbg gene is described in FIG. 4.

The invention also pertains to a genetic screening method to identify individuals susceptible to developing hypercortisolism and associated pathologies with polymorphic markers for selecting or negative selecting breeding animals, preferably pigs, having a high probability of developing hypercortisolism and a high fattening rate.

We detected, from sequences of the exons of the Cbg gene in F1 and F0 pigs, the following polymorphisms:
transition G→T corresponding to position 133 of SEQ ID NO. 1,
transition C→T corresponding to position 134 of SEQ ID NO. 1,
transition C→C corresponding to position 539 of SEQ ID NO. 1,
transition A→G corresponding to position 620 of SEQ ID NO. 1,
transition G→A corresponding to position 626 of SEQ ID NO. 1,
transition C→T corresponding to position 859 of SEQ ID NO. 1,
transition C→T corresponding to position 866 of SEQ ID NO. 1,
transition A→C corresponding to position 882 of SEQ ID NO. 1,
transition G→C corresponding to position 890 of SEQ ID NO. 1,
transition C→T corresponding to position 960 of SEQ ID NO. 1,
transition G→A corresponding to position 1008 of SEQ ID NO. 1,
transition T→C corresponding to position 42 of SEQ ID NO. 6,
transition C→T corresponding to position 49 of SEQ ID NO. 6,
transition C→T corresponding to position 75 of SEQ ID NO. 7.

The sequences SEQ ID NO. 6 and 7 correspond respectively to part 5′ and part 3′ of the C intron of the porcine Cbg gene. The invention, therefore, pertains to a genetic screening method to identify individuals susceptible to develop hypercortisolism and associated pathologies with polymorphic markers in which the alleles of the polymorphic marker as defined above have one of the mutations described above.

The invention also involves a kit for implementing the aforementioned method enabling testing of genetic markers of hypercortisolism from a DNA sample. Such a kit comprises a pair of nucleotide primers as defined above used with commercially available PCR amplification reagents. The kit can also include negative and positive controls of the reactions and the markers.

The invention also pertains to a method to identify substances capable of modulating the expression of the Cbg gene and/or its synthesis with the therapeutic goal of reducing a hypercortisolism. In fact, CBG protein of wild or mutant type can be used for an in vitro screening for compounds capable of modifying the binding of CBG to cortisol and/or corticosterone. The invention, therefore, relates to a method for identifying substances capable of modulating the function of CBG consisting of measuring by any suitable technique binding of the compound to (wild or mutant) CBG. This can be a technique using the large-scale screening methods described in the literature such as, for example, “High Throughput Screening: The Discovery of Bioactive Substances”, J P Delvin (editor), Marcel Dekker Inc., New York (1997).

Binding activity between CBG and an active compound can be determined, for example, by a radiobinding test in which the binding capacity and the affinity of the test compounds are evaluated on the basis of their radioactive cortisol displacement capacity. The source of CBG is obtained, for example, by transfection of a vector containing cDNA of the Cbg gene in cultured cells. Since the protein is secreted, the radiobinding test can be performed on the culture medium. The compounds demonstrating efficacy in competition with cortisol are selected.

The invention also pertains to the use of animals overexpressing the Cbg gene or expressing a mutant of this gene as a model for comprehending the mechanisms of action of CBG on the corticotropic axis and/or for screening for compounds capable of modulating the expression of CBG. The invention, moreover, pertains to transgenic animals whose transgene contains a nucleic acid sequence contained in the Cbg gene or the adjacent 3′ and 5′ sequences preferably distanced apart by no more about than 100 kb. The selected sequences preferably code for a polypeptide identical to or homologous with the protein CBG.

“Homologous” as sometimes hereinafter used means a degree of homology to the isolated and described domains in excess of about 70%, most preferably in excess of about 80%, and even more preferably in excess of about 90%, about 95% or about 99%. Locating the parts of these sequences that are not critical may be time consuming, but is routine and well within the skill in the art. Sequence identity or homology as sometimes used herein, indicates that a nucleotide sequence or an amino acid sequence exhibits substantial structural or functional equivalence with another nucleotide or amino acid sequence. Any structural or functional differences between sequences having substantial sequence identity or homology will be de minimis; that is, they will not affect the ability of the sequence to function as indicated in the desired application. Differences may be due to inherent variations in codon usage among different species, for example. Structural differences are considered de minimis if there is a significant amount of sequence overlap or similarity between two or more different sequences or if the different sequences exhibit similar physical characteristics even if the sequences differ in length or structure. Such characteristics include, for example, ability to maintain expression and properly fold into the proteins conformational native state, hybridize under defined conditions, or demonstrate a well defined immunological cross-reactivity, similar biopharmaceutical activity, etc. Each of these characteristics can readily be determined by the skilled practitioner in the art using known methods.

The transgenic animals are obtained by microinjection in animal embryos (for example, mice, rat, pigs and the like) of a nucleic acid contain the coding sequence of the Cbg gene (for example, SEQ ID NO. 1) as well as regulatory sequences enabling its overexpression in the target tissue (in this case, the liver) in accordance with conventional practice in this technology.

These animals can be used as technical models for understanding the mechanisms of action of CBG on the corticotropic axis and the pathologies associated with obesity, the inflammatory and autoimmune responses, aging—particularly cognitive aging and drug addictions. These animals can also be used for screening for compounds capable of modulating the function of CBG. Screening of compounds can be performed by administration to the animal of the test compound followed by measurement of the changes in the animal in relation to corticotropic function by conventional methods.

The invention also pertains to a method for screening for compounds capable of modulating expression of the Cbg gene and/or its synthesis and/or its binding to cortisol with the therapeutic goal of reducing a hypercortisolism and, as a consequence of curing pathologies linked to this hypercortisolism such as obesity, constitutive sensitivity to inflammatory and autoimmune reactions, as well as the pathologies of aging and sensitization to drug abuse. This method comprises producing the protein CBG from cultured cells, for example, HepG2 cells and testing the compound versus the protein. This screening is advantageously a large-scale screening.

The invention also pertains to a method for screening for a compound capable of modulating expression of the Cbg gene and/or its synthesis and/or its binding to cortisol, comprising in vivo screening on a transgenic animal as described above a compound identified in vitro in accordance with the previously described screening method.

Identification of the genetic markers according to the invention makes available new tools for implementing genetic tests for CBG to evaluate constitutive hypercortisolism and, consequently, vulnerability to the previously specified pathologies, notably obesity. Such a test is also useful for negative selection of breeding animals exhibiting a constitutive hypercortisolism.

As an example, such a method comprises PCR amplification of a region of the DNA of the sample comprising all or part of the Cbg gene and analysis of this region to identify the presence of at least one mutation responsible for a hypercortisolism and susceptible to have been identified by the previously described method.

The invention, therefore, also pertains to the use of the above method for diagnosing a hypercortisolism or a predisposition to a hypercortisolism in a subject, especially a human subject, enabling identification of a dysfunction of the corticotropic axis and, thus, a disease or a predisposition to a disease linked to this axis such as obesity, constitutive sensitivity to inflammatory and autoimmune reactions, or pathologies of aging (cognitive aging in particular) or sensitization to drugs of abuse.

Finally, the drug pertains to identifying agonist or antagonist compounds of CBG and which are therefore capable of acting directly on the CBG levels or the affinity of CBG for cortisol which indirectly reduces the corticosteroid levels.

### EXAMPLES

### 1. Test Methods

### 1) Cartography on Irradiated Hybrids

Reactions were performed independently in duplicate on an ImpRH panel (Yerle et al., 1998). The PCR products were analyzed on 2% agarose gels in 1×TBE buffer after staining with ethidium bromide. A third amplification was performed on the clones for which discordant results were obtained. The vectors of the amplification results were then submitted to the ImpRH data bank (Milan et al., 2000).

### 2) FISH Cartography

The chromosomes in metaphase were obtained from peripheral blood lymphocyte cultures. The metaphases were marked in G bands using a G-T-G technique prior to hybridization to identify the chromosomes, and the images of the best metaphases: were taken with a video printer as previously described (Yerle et al, 1992).

In-situ hybridization experiments were performed in accordance with Yerle et al. (Yerle et al., 1992) with some modifications (Sun et al., 1999).

### 3) Genetic Link Analysis

Distribution of data according to a normal law was first verified. The four characters having normal logarithmic distributions and the data were transformed into logarithmic scores prior to analysis. QTL cartography was performed using multipoint maximum likelihood techniques. A statistical test defined as the ratio of the likelihoods under the hypotheses of one (H1) versus none (H0) of QTL linked to the set of markers considered was calculated at each position (each cM) along the chromosome. The marker map of the chromosome 7 employed was calculated from the genotypes of more than 1100 pigs by Bidanel et al. (2000). According to the H1 hypothesis, a QTL with a gene substitution effect for each father and mother was adjusted to the data. Other details on the probability calculation procedures can be found in Bidanel et al. (2000). Estimations of the mean effects of substitution were calculated at each position with the highest probability ratio.

Thresholds of significance along the chromosomes were determined empirically by simulating the data assuming an infinitesimal model and a normal distribution of the performances. A total of 50,000 simulations were performed for each character. The level of significance of the chromosome test P_{c} corresponding to a probability test of the entire genome P_{g} was obtained by using the Bonferroni correction, i.e., as a solution of: P_{g}=1−(1−P_{c})¹⁹ which yields P_{c}=0.0027 and 0.000054, respectively, for significant (P_{g}=0.05) and very significant levels (Knott et al., 1998).

### 4) Screening the BAC DNA Data Bank

BAC clones were isolated by three-dimensional PCR screening of a porcine data bank of BAG clones as previously described (Rogel-Gaillard et al., 1999). The clone BAC 383F4 containing the porcine CBG sequence was cloned using a pair of primers established from the sequence of exon 2 of human CBG:

| | | |
|---|---|---|
| FW: | ACACCTGTCTTCTCTGGCTG | (SEQ ID NO.4) |
| REV: | ACAGGCTGAAGGCAAAGTC. | (SEQ ID NO.5) |

The PCR were performed on 35 cycles of 30 seconds at 94° C., 30 seconds at 56° C., 30 seconds at 72° C., in a reaction volume of 20 μl containing 0.2 mM of each dNTP, 1.5 mM of MgCl₂, 8 pM of each primer, 2 U of Taq DNA polymerase and reaction buffer (Perkin-Elmer, Roche).

### 5) Sequencing

Sequence reactions were performed with the kit “Prism AmpliTaq FS diChloroRhodamine Dye Terminators” (Perkin-Elmer) on a PE 970 automatic sequencer.

The binding capacity of CBG and its affinity for cortisol were measured at 4° C. by a solid phase fixation test using a Concavalin A-Sepharose column (Pugeat et al., 1984). The association equilibrium constant (Ka) and the capacity of CBG for cortisol were calculated by a Scatchard analysis using “bound” as the quantity of cortisol specifically fixed to the glycoproteins adsorbed on the gel and “free” as the concentration of cortisol in the aqueous phase.

### 6) Statistics

The correlation matrices and Student's t test were performed using Version 5 of the Statistica program.

### II. Results

### 1) Comparative Cartography Allows us to Propose the Cbg Gene as Candidate for the QTL of Hypercortisolism

Goureau et al. (2000) described correspondence of the segments of the human and porcine chromosomes using a bidirectional chromosomal paint. The cortisol QTL flanked by the markers S0101 and Sw764 were localized on the porcine region 7q2.4-7q2.6. Among the genes localized on the homologous human region (Hsap14q), the gene coding for CBG localized on Hsap14q32.1 (Billingsley et al., 1993) attracted attention. In fact, 90% of the plasma cortisol is fixed to CBG which is a glycoprotein synthesized by the liver. Since only the free cortisol is active, CBG has a major role in the bioavailability of cortisol. Thus, the Cbg gene constitutes a good functional candidate for this QTL associated with cortisol levels.

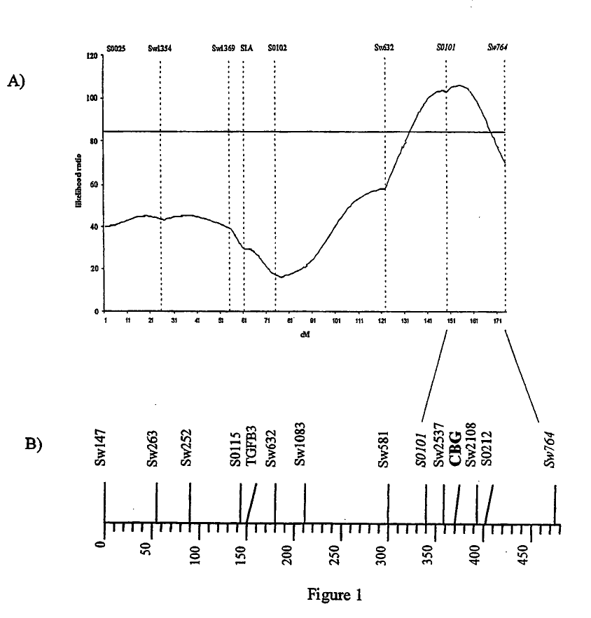

Since the Cbg has been cloned in humans, monkeys, sheep and mice (Hammond et al., 1987; Hammond et al., 1994; Berdusco et al., 1993; Orava et al., 1994), it was possible to align the different available sequences using the Multalin program (Corpet, 1988) and prepare consensus oligonucleotide primers from the exon 2 to obtain a PCR fragment of porcine Cbg. After verification of the strong homology of the sequence of the PCR fragment with the Cbg gene of other species, the primers were used for mapping the porcine Cbg gene using a panel of irradiated hybrids (Yerle et al., 1988). It was then found that the porcine Cbg gene was located between the markers S0101 and SW764, like the cortisol QTL as shown in FIG. 1.

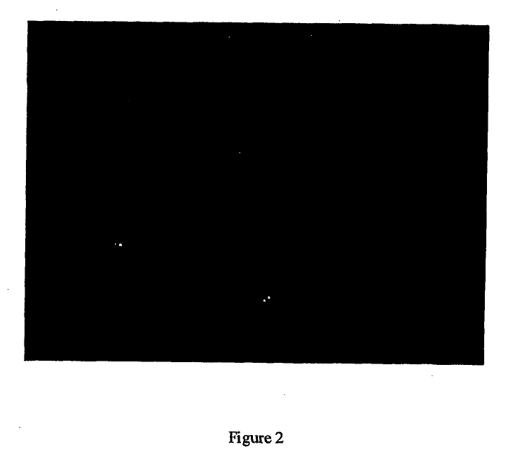

This chromosomal localization was confirmed by fluorescent in-situ hybridization (FISH). First, a porcine genomic BAC data bank was screened by PCR with the primers amplifying the exon 2 of the porcine Cbg gene. A 150-kb clone, named BAC 383F4, containing the totality of the genomic sequence of the porcine Cbg gene was obtained. This BAC clone was used as s probe for mapping the porcine Cbg gene by FISH on a range of chromosomes in metaphase. It was confirmed that the porcine Cbg gene is found at 7q26 of the chromosome as shown in FIG. 2.

### 2) The CBG Binding Capacity is Genetically Linked to the Markers Flanking the QTL of Hypercortisolism

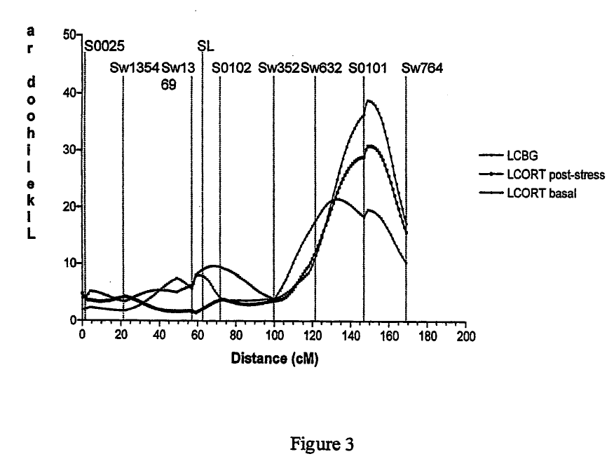

The binding capacity of CBG to cortisol was measured in the plasma of 81 F2 pigs from the original crossing, all descendents of the F1 pig #911045. As expected, a strong correlation was found between this measurement and the cortisol level (r=0.57). The genetic link between this new phenotypic measurement and the Ssc7 markers was evaluated. FIG. 3 shows that a strong genetic link was detected in the same region as for the cortisol QTL. The maximal probability is even higher with the CBG values (p<5·10⁻⁴) which reinforces the implication of the Cbg gene in this QTL.

### 3) The CBG Binding Capacity is Different Between LW and MS Pigs

At the protein level, the capacity of binding to cortisol and the affinity constant of CBG were compared between the LW and Meishan pigs by radiobinding studies. No difference in affinity was found between the two breeds of pigs. However, as seen in Table 1 below, the maximum binding capacity was on average 1.6 times higher in the Meishan pigs compared to the LW pigs (p<0.001).

TABLE 1

| | Large White | Meishan | t | |
|---|---|---|---|---|
| | n = 12 | n = 12 | (Student's test) | p |
| Bmax (nM/l of | 46.89 ± 4.83 | 74.38 ± 5.95 | 3.996 | <0.001 |
| blood) | | | | |
| Kd (nM) | 0.38 ± 0.05 | 0.46 ± 0.05 | 1.038 | <0.3 |

### 4) Identification of a Mutation in the Cbg Gene

The clone BAC 383F4 enabled identification of the genomic organization and the sequence of the Cbg gene which had never been previously cloned. The porcine Cbg gene contains 5 exons with an AUG codon in exon 2 as is the case in other species. At the level of amino acids, the inventors found 66% and 80% of homology between porcine CBG and those of humans and sheep, respectively.

The exons and 900 pb of the promoter region of an F1 animal, of its LW father and its Meishan mother were sequenced to investigate mutations. It was concluded that the F1 pig (#911045) had to be heterozygous to the QTL because there was a significant difference between the average cortisol levels between the progenitor who had received one or the other allele of the marker S0101 flanking the QTL. Consequently, the Meishan mother should have at least one allele different from the LW father at the level of the mutation under consideration. A mutation of this type was identified in exon 2 of the F1 pig #9110045. In position +15 from the ATG starting codon, this animal is heterozygous with a punctiform mutation G→T on one allele (FIG. 4). This G→T substitution corresponds to codon 15, change of a serine into an isoleucine in the signal protein of the CBG protein. The PCR amplification test was optimized with the following parameters:

(SEQ ID NO: 8)

| | |
|---|---|
| Sense primer: | 5′-CCCTGTATGCCTGTCTCCTC-3′, |

(SEQ ID NO: 9)

| | |
|---|---|
| Antisense primer: | 5′-CCCTGCTCCAAGAACAAGTCC-3′, |

PCR conditions: 1×PCR buffer (Promega), 1.5 mM MgC₂, 100 μM dNTP, 10 μmol of each primer, 0.4 U Taq polymerase (Promega).

Thermocycler Program:

1) 96° C.: 5 minutes

2) 92° C.: 30 seconds

3) 60° C.: 11 minute

4) 72° C. 30 seconds

5) step 2) 3) and 4) 34 times

6) 72° C.: 2 minutes.

### BIBLIOGRAPHIC REFERENCES

Armario, A., Gavalda, A., and Marti, J. (1995). Comparison of the behavioural and endocrine response to forced swimming stress in five inbred strains of rats. Psychoneuroendocrinology 20:879-890.
Berdusco, E. T., Hammond, G. L., Jacobs, R. A., Grolla, A., Akagi, K, Langlois, D., and Challis, J. R. (1993). Glucocorticoid-induced increase in plasma corticosteroid-binding globulin levels in fetal sheep is associated with increased biosynthesis and alterations in glycosylation. Endocrinology 132:2001-2008.
Bidanel, J. P., Milan, D., Chevalet, C., Woloszyn, N., Bourgeois, F., Caritez, J. C., Gruand, J., LeRoy, P., Bonneau, M., Lefaucheur, L., Mourot, J., Prunier, M., Désautés, C., Mormède, P., Renard, C., Vaiman, M., Robic, A., Gellin, J., and Ollivier, L. (1998). Detection of locus with quantitative effects in the cross between the pig breeds Large White and Meishan. Experimental device and initial results. Journées Rech Porcine en France 30:117-125.
Bidanel, J. P., Milan, D., Iannuccelli, N., Amigues, Y., Bosher, M.-Y., Bourgeois, F., Caritez, J.-C., Gruand, J., LeRoy, P., Lagand, H. B. M., Lefaucheur, L., Mourot, J., Prunier, A., Désautés, C., Mormède, P., Renard, C., Vaiman, M., Robic, A., Gellin, J., Ollivier, L., and Chevalet, C. (2000). Detection of locus with quantitative effects in the cross between the pig breeds Large White and Meishan: Results and Perspectives. Journées Rech Porcine en France 32:369-383.
Billingsley, G. D., Walter, M. A., Hammond, G. L., and Cox, D. W. (1993). Physical mapping of four serpin genes: alpha 1-antitrypsin, alpha 1-antichymotrypsin, corticosteroid-binding globulin, and protein C inhibitor, within a 280-kb region on chromosome 14q32.1. Am J Hum Genet. 52:343-353.
Buemann, B., Vohl, M. C., Chagnon, M., Chagnon, Y. C., Gagnon, J. Perusse, L., Dionne, F., Despres, J. P., Tremblay, A., Nadeau, A., and Bouchard, C. (1.997). Abdominal visceral fat is associated with a BclI restriction fragment length polymorphism at the glucocorticoid receptor gene locus. Obes Res 5:186-192.
Corpet, F. (1988). Multiple sequence alignment with hierarchical clustering. Nucleic Acids Res 16:10881-10890.
Crave, J. C., Lejeune, H., Brebant, C., Baret, C., and Pugeat, M. (1995). Differential effects of insulin and insulin-like growth factor I on the production of plasma steroid-binding globulins by human hepatoblastoma-derived (Hep G2) cells. J Clin Endocrinol Metab 80:1283-1289.
Désautés, C., Bidanel, J. P., and Mormède, P. (1997). Genetic study of behavioral and pituitary-adrenocortical reactivity in response to an environmental challenge in pigs. Physiol Behav 62:337-345.
Désautés, C., Sarrieau, A., Caritez, J. C., and Mormède, P. (1999). Behavior and pituitary-adrenal function in Large White and Meishan pigs. Domestic animal endocrinology 16:193-205.
Emptoz-Bonneton, A., Cousin, P., Seguchi, K., Avvakumov, G. V., Bully, C., Hammond, G. L., and Pugeat, M. (2000). Novel human corticosteroid-binding globulin variant with low cortisol-binding affinity. J Clin Endocrinol Metab 85:361-367.
Fernandez-Real, J. M., Grasa, M., Casamitjana, R., Pugeat, M., Barret, C., and Ricart, W. (1999). Plasma total and glycosylated corticosteroid-binding globulin levels are associated with insulin secretion. J Clin Endocrinol Metab 84:3192-3196.
Fernandez-Real, J. M., Grasa, M., Casamitjana, R., and Ricart, W. (2000). The insulin resistance syndrome and the binding capacity of cortisol binding globulin (CBG) in men and women. Clin Endocrinol (Oxf) 52:93-99.
Goureau, A., Vignoles, M., Pinton, P., Gellin, J., and Yerle, M. (2000). Improvement of comparative map between porcine chromosomes 1 and 7 and human chromosomes 6, 14, and 15 by using human YACs. Mamm Genome 11:796-799.
Hammond, G. L. (1995). Potential functions of plasma steroid-binding proteins. Trends in Endocrinology and Metabolism 6:298-304.
Hammond, G. L., Smith, C. L., Goping, L. S., Underhill, D. A., Harley, M. J., Reventos, J., Musto, N. A., Gunsalus, G. L. and Bardin, C. W. (1987). Primary structure of human corticosteroid binding; globulin, deduced from hepatic and pulmonary cDNAs, exhibits homology with serine protease inhibitors. Proc Natl Acad Sci USA 84:51.53-5157.
Hammond, G. L., Smith, C. L., Lahteenmaki, P., Grolla, A., Warmels-Rodenhiser, S., Hodgert, H., Murai, J. T., and Siiteri, P. K. (1994). Squirrel monkey corticosteroid-binding globulin: primary structure and comparison with the human protein. Endocrinology 134:891-898.
Hammond, G. L., Smith, C. L., Paterson, N. A., and Sibbald, W. J. (1990). A role for corticosteroid-binding globulin in delivery of cortisol to activated neutrophils. J Clin Endocrinol Metab 71:34-39.
Kirschbaum, C., Wust, S., Faig, H. G., and Hellhammer, D. H. (1992). Heritability of cortisol responses to human corticotropin-releasing hormone, ergometry, and psychological stress in humans. J Clin Endocrinol Metab 75:1526-1530.
Knott, S. A., Marklund, L., Haley, C. S., Andersson, K., Davies, W., Ellegren, H., Fredholm, M., Hansson, I., Hoyheim, B., Lundstrom, K., Moller, M., and Andersson, L. (1998). Multiple marker mapping of quantitative trait loci in a cross between outbred wild boar and large white pigs. Genetics 149:1069-1080.
Linkowski, P., Van Onderbergen, A., Kerkhofs, M., Bosson, D., Mendlewicz, J., and Van Cauter, E. (1993). Twin study of the 24-h cortisol, profile: evidence for genetic control of the human circadian clock. Am J Physiol 264:E173-E181.
Lupien, S. J., deLeon, M., deSanti, S., Convit, A., Tarshish, C., Nair, N. P., Thakur, M., McEwen, B. S., Hauger, R. L., and Meaney, M. J. (1998). Cortisol levels during human aging predict hippocampal atrophy and memory deficits [see comments] [published erratum appears in Nat Neurosci 1998 August; 1(4):329]. Nat Neurosci 1:69-73.
Marissal-Arvy, N., Mormede, P., and Sarrieau, A. (1999). Strain differences in corticosteroid receptor efficiencies and regulation in Brown Norway and Fischer 344 rats. J Neuroendocrinol 11:267-273.
Marissal-Arvy, N., Ribot, E., Sarrieau, A., and Mormede, P. (2000). Is the mineralocorticoid receptor in Brown Norway rats constitutively active? J Neuroendocrinol 12:576-588.
Meikle, A. W., Stringham, J. D., Woodward, M. G., and Bishop, D. T. (1988). Heritability of variation of plasma cortisol levels. Metabolism 37:514-517.
Milan, D., Hawken, R., Cabau, C., Leroux, S., Genet, C., Lahbib, Y., Tosser, G., Robic, A., Hatey, F., Alexander, L., Beattie, C., Schook, L., Yerle, M., and Gellin, J. (2000). IMpRH server: an RH mapping server available on the Web [In Process Citation]. Bioinformatics 16:558-559.
Moisan, M. P., Courvoisier, H., Bihoreau, M. T., Gauguier, D., Hendley, E. D., Lathrop, M., James, M. R., and Mormède, P. (1996). A major quantitative trait locus influences hyperactivity in the WKHA rat. Nat Genet. 14:471-473.
Mormède, P., Dantzer, R., Bluthe, R.-M., and Caritez, J.-C. (1984). Differences in adaptive abilities of three breeds of chinese pigs. Genet Sel Evol 16:85-102.
Orava, M., Zhao, X. F., Leiter, E., and Hammond, G. L. (1994). Structure and chromosomal location of the gene encoding mouse corticosteroid-binding globulin: strain differences in coding sequence and steroid-binding activity. Gene 144:259-264.
Piazza, P. V. and LeMoal, M. (1998). The role of stress in drug self-administration. Trends Pharmacol Sci 19:67-74.
Pugeat, M. M., Chrousos, G. P., Nisula, B. C., Loriaux, D. L., Brandon, D., and Lipsett, M. B. (1984). Plasma cortisol transport and primate evolution. Endocrinology 115:357-361.
Rogel-Gaillard, C., Bourgeaux, N., Billault, A., Vaiman, M., and Chardon, P. (1999). Construction of a swine BAC library: application to the characterization; and mapping of porcine type C endoviral elements. Cytogenet Cell Genet. 85:205-211.
Rosmond, R., Dallman, M. F., and Bjorntorp, P. (1998). Stress-related cortisol secretion in men: relationships with abdominal obesity and endocrine, metabolic and hemodynamic abnormalities [see comments]. J Clin Endocrinol Metab 83:1853-1859.
Sambrook, J., Fritsch, E. F., and Maniatis, T. (1989). Molecular cloning: A laboratory manual, 2^{nd} edition. Cold Spring Harbor Laboratory Press, Cold Spring Harbor, N.Y.
Smith, C. L. and Hammond, G. L. (1991). An amino acid substitution in biobreeding rat corticosteroid binding globulin results in reduced steroid binding affinity. J Biol Chem 266:18555-18559.
Smith, C. L., Power, S. G., and Hammond, G. L. (1992). A Leu—His substitution at residue 93 in human corticosteroid binding globulin results in reduced affinity for cortisol. J Steroid Biochem Mol Biol 42:671-676.
Sternberg, E. and Gold, P. (1997). Emotions and disease. From balance of humors to balance of molecule. Nature Medicine 3:264-267.
Sun, H. F., Ernst, C. W., Yerle, M., Pinton, P., Rothschild, M. F., Chardon, P., Rogel-Gaillard, C., and Tuggle, C. K. (1999). Human chromosome 3 and pig chromosome 13 show complete synteny conservation but extensive gene-order differences. Cytogenet Cell Genet. 85:273-278.
Torpy, D. J., Bachmann, A. W., Grice, J. E., Fitzgerald, S. P., Phillips, P. J., Whitworth, J. A., Jackson, R. V. (2001) in press J Clin Endocrinol Metab 86 (6).
Van Baelen, H., Brepoels, R., and DeMoor, P. (1982). Transcortin Leuven: a variant of human corticosteroid-binding, globulin with decreased cortisol-binding affinity. J Biol Chem 257:3397-3400.
Warden, C. H., Fisler, J. S., Shoemaker, S. M., Wen, P. Z., Svenson, K. L., Pace, M. J., and Lusis, A. J. (1995). Identification of four chromosomal loci determining obesity in a multifactorial mouse model. J Clin Invest 95:1545-1552.
Yerle, M., Galman, O., Lahbib-Mansais, Y., and Gellin, J. (1992). Localization of the pig luteinizing hormone/choriogonadotropin receptor gene (LHCGR) by radioactive and nonradioactive in situ hybridization. Cytogenet Cell Genet. 59:48-51.
Yerle, M., Pinton, P., Robic, A., Alfonso, A., Palvadeau, Y., Delcros, C., Hawken, R., Alexander, L., Beattie, C., Schook, L., Milan, D., and Gellin, J. (1998). Construction of a whole-genome radiation hybrid panel for high-resolution gene mapping in pigs. Cytogenet Cell Genet. 82:182-188.

1-14. (canceled)

15. A method for identifying polymorphic markers associated with a hypercortisolism phenotype comprising:
comparing nucleic acid sequences, from multiple individuals, comprising all or part of a Cbg gene; and
identifying mutations in the Cbg gene or sequences adjacent to it.

16. The method according to claim 15, wherein the nucleic acid sequences are genomic DNA sequences comprising a part of the Cbg gene or an adjacent 3′ or 5′ sequence distanced apart by no more than about 100 kb.

17. A polymorphic marker responsible for a hypercortisolism phenotype comprising all or part of a nucleic acid sequence comprising a Cbg gene or adjacent 3′ or 5′ sequences distanced apart by no more than about 100 kb.

18. The marker according to claim 17, selected from the group consisting of microsatellites, insertion/deletion polymorphisms, restriction fragment length polymorphisms (RFLP) and single nucleotide polymorphisms (SNP).

19. A nucleotide primer comprising from about 5 to about 50 successive nucleotides of a sequence of the Cbg gene or the adjacent 3′ or 5′ sequences distanced apart by no more than about 100 kb, flanking a marker according to claim 17.

20. A genetic screening method for identifying individuals predisposed to develop hypercortisolism and associated pathologies with polymorphic markers comprising:
i) purifying genomic DNA from an individual,
ii) amplifying a locus containing a polymorphic marker by PCR from the DNA, wherein alleles of the polymorphic marker have at least one mutation indicative of a predisposition to hypercortisolism selected from the group consisting of
transition G→T corresponding to position 133 of SEQ ID NO. 1,
transition C→T corresponding to position 134 of SEQ ID NO. 1,
transition C→T corresponding to position 539 of SEQ ID NO. 1,
transition G→A corresponding to position 620 of SEQ ID NO. 1,
transition G→A corresponding to position 626 of SEQ ID NO. 1,
transition C→T corresponding to position 859 of SEQ ID NO. 1,
transition T→C corresponding to position 866 of SEQ ID NO. 1,
transition A→G corresponding to position 882 of SEQ ID NO. 1,
transition G→C corresponding to position 890 of SEQ ID NO. 1,
transition C→T corresponding to position 960 of SEQ ID NO. 1,
transition G→A corresponding to position 1008 of SEQ ID NO. 1,
transition T→C corresponding to position 42 of SEQ ID NO. 6,
transition C→T corresponding to position 49 of SEQ ID NO. 6, and
transition C→T corresponding to position 75 of SEQ ID NO. 7; and
iii) detecting one or more of said allele(s) of the polymorphic marker in the amplified DNA, wherein the individual is identified as predisposed to develop hypercortisolism when one or more alleles of the polymorphic marker defined in step (ii) is detected.

21. The method according to claim 20, wherein the mutation is transition G→A corresponding to position 1008 of SEQ ID NO. 1.

22. The method according to claim 20, wherein the subject is a pig.

23. A kit for testing genetic markers of hypercortisolism from a DNA sample comprising:
a pair of nucleotide primers according to claim 19;
PCR reagents; and
one of negative and positive controls of reactions and the markers.

24. A method of diagnosing a hypercortisolism or a predisposition to a hypercortisolism in a subject, enabling identification of a dysfunction of the corticotropic axis and a disease or a predisposition to a disease linked to this axis comprising:
i) purifying genomic DNA from an individual,
ii) amplifying a locus containing a polymorphic marker by PCR from the DNA, wherein alleles of the polymorphic marker have at least one mutation indicative of a predisposition to hypercortisolism selected from the group consisting of
transition G→T corresponding to position 133 of SEQ ID NO. 1,
transition C→T corresponding to position 134 of SEQ ID NO. 1,
transition C→T corresponding to position 539 of SEQ ID NO. 1,
transition G→A corresponding to position 620 of SEQ ID NO. 1,
transition G→A corresponding to position 626 of SEQ ID NO. 1,
transition C→T corresponding to position 859 of SEQ ID NO. 1,
transition T→C corresponding to position 866 of SEQ ID NO. 1,
transition A→G corresponding to position 882 of SEQ ID NO. 1,
transition G→C corresponding to position 890 of SEQ ID NO. 1,
transition C→T corresponding to position 960 of SEQ ID NO. 1,
transition G→A corresponding to position 1008 of SEQ ID NO. 1,
transition T→C corresponding to position 42 of SEQ ID NO. 6,
transition C→T corresponding to position 49 of SEQ ID NO. 6, and
transition C→T corresponding to position 75 of SEQ ID NO. 7;
iii) detecting one or more of said allele(s) of the polymorphic marker in the amplified DNA, and
iv) diagnosing hypercortisolism or a predisposition to hypercortisolism in the subject when one or more of said alleles is detected.

25. The method according to claim 24, wherein the mutation is transition G A corresponding to position 1008 of SEQ ID NO. 1.

26. The method according to claim 32, wherein the disease is selected from the group consisting of obesity, constitutive sensitivity to inflammatory and autoimmune reactions, pathologies of aging and sensitization to drugs of abuse.

27. A transgenic animal transgene containing of the nucleic sequences of claim 17.

28. A transgenic animal overexpressing sequences according to claim 17 and coding for a polypeptide identical to or homologous with the protein CBG.

29. A method for identifying a compound that modulates a function of CBG protein and reduces a hypercortisolism of a subject comprising:
binding the compound to the CBG protein;
determining cortisol displacement capacity between the CBG protein and the compound; and
selecting compounds exhibiting efficacy relative to cortisol.

30. A method of identification of a dysfunction of the corticotropic axis comprising:
i) purifying genomic DNA from an individual,
ii) amplifying a locus containing a polymorphic marker by PCR from the DNA,
wherein the alleles of the polymorphic marker have a mutation selected from the group consisting of:
transition G→T corresponding to position 133 of SEQ ID NO. 1,
transition C→T corresponding to position 134 of SEQ ID NO. 1,
transition C→T corresponding to position 539 of SEQ ID NO. 1,
transition G→A corresponding to position 620 of SEQ ID NO. 1,
transition G→A corresponding to position 626 of SEQ ID NO. 1,
transition C→T corresponding to position 859 of SEQ ID NO. 1,
transition T→C corresponding to position 866 of SEQ ID NO. 1,
transition A→G corresponding to position 882 of SEQ ID NO. 1,
transition A→C corresponding to position 890 of SEQ ID NO. 1,
transition C→T corresponding to position 960 of SEQ ID NO. 1,
transition G→A corresponding to position 1008 of SEQ ID NO. 1,
transition T→C corresponding to position 42 of SEQ ID NO. 6,
transition C→T corresponding to position 49 of SEQ ID NO. 6, and
transition C→T corresponding to position 75 of SEQ ID NO. 7; and
iii) detecting said allele(s) of the polymorphic marker in the amplified DNA;
wherein a dysfunction of the corticotropic axis in the subject is identified when said allele(s) of the polymorphic marker is detected in step (iii).

31. The method according to claim 30, wherein the mutation is transition G→A corresponding to position 1008 of SEQ ID NO. 1.

32. A method of identification of a disease or a predisposition to develop a disease, comprising:
i) purifying genomic DNA from an individual,
ii) amplifying a locus containing a polymorphic marker by PCR from the DNA, wherein the alleles of the polymorphic marker have a mutation selected from the group consisting of
transition G→T corresponding to position 133 of SEQ ID NO. 1,
transition C→T corresponding to position 134 of SEQ ID NO. 1,
transition C→T corresponding to position 539 of SEQ ID NO. 1,
transition G→A corresponding to position 620 of SEQ ID NO. 1,
transition G→A corresponding to position 626 of SEQ ID NO. 1,
transition C→T corresponding to position 859 of SEQ ID NO. 1,
transition T→C corresponding to position 866 of SEQ ID NO. 1,
transition A→G corresponding to position 882 of SEQ ID NO. 1,
transition G→C corresponding to position 890 of SEQ ID NO. 1,
transition C→T corresponding to position 960 of SEQ ID NO. 1,
transition G→A corresponding to position 1008 of SEQ ID NO. 1,
transition T→C corresponding to position 42 of SEQ ID NO. 6,
transition C→T corresponding to position 49 of SEQ ID NO. 6, and
transition C→T corresponding to position 75 of SEQ ID NO. 7; and
iii) detecting said allele(s) of the polymorphic marker in the amplified DNA;
wherein the disease or predisposition to develop the disease is identified in the subject when said allele(s) of the polymorphic marker is detected in step (iii).

33. The method according to claim 32, wherein the mutation is transition G A corresponding to position 1008 of SEQ ID NO. 1.
